(19) United States
(12) Patent Application Publication
(10) Pub. No.: US 2010/0291597 A1
(43) Pub. Date: Nov. 18, 2010

(54) SECRETOGRANIN AND VGF PEPTIDE BIOMARKERS AND USES THEREOF

(75) Inventors: Sabine Bahn, Cambridge (GB); Jeffrey T.-J. Huang, Dundee (GB)

Correspondence Address: FOLEY AND LARDNER LLP;SUITE 500, 3000 K STREET NW, WASHINGTON, DC, 20007, US

(73) Assignee: CAMBRIDGE ENTERPRISE LIMITED, Cambridge (GB)

(21) Appl. No.: 12/307,243

(22) PCT Filed: Aug. 15, 2007
(86) PCT No.: PCT/GB2007/003090

§ 371 Date: Jul. 28, 2010

(30) Foreign Application Priority Data
Aug. 16, 2006 (GB) 0616230.9

(51) Int. Cl. G01N 33/53; G01N 33/68

(52) U.S. Cl. 435/7.92; 436/86; 435/7.1

(57) ABSTRACT

Secretogranin II and VGF peptides are biomarkers for major depressive disorder. They are useful in methods of diagnosing, monitoring and screening.

### FIELD OF THE INVENTION

The present invention relates to peptide biomarkers for major depressive disorders. Biomarkers and methods in which they are employed can be used to assist diagnosis or to assess onset and development of major depressive disorder. The invention also relates to use of biomarkers in clinical screening, assessment of prognosis, evaluation of therapy, and for drug screening and drug development in the field of major depressive disorder.

### BACKGROUND OF THE INVENTION

The Diagnostic and Statistical Manual of Mental Disorders fourth edition (DSM IV) published by the American Psychiatric Association, Washington D.C., 1994, has proven to be an authoritative reference handbook for health professionals both in the United Kingdom and in the United States in categorizing and diagnosing mental health problems. This describes the diagnostic criteria, subtypes, associated features and criteria for differential diagnosis of mental health disorders, including major depressive disorder.

According to the DSM-IV, a person who suffers from a major depressive disorder must either have a depressed mood or a loss of interest or pleasure in daily activities consistently for at least a two week period. This mood must represent a change from the person's normal mood; social, occupational, educational or other important functioning must also be negatively impaired by the change in mood. A depressed mood caused by substances (such as drugs, alcohol or medications) is not considered a major depressive disorder, nor is one which is caused by a general medical condition. Major depressive disorder cannot be diagnosed if a person has a history of manic, hypomanic, or mixed episodes (e.g., a bipolar disorder) or if the depressed mood is better accounted for by schizoaffective disorder and is not superimposed on schizophrenia, a delusional or psychotic disorder. Further, the symptoms are not better accounted for by bereavement, i.e., after the loss of a loved one, the symptoms persist for longer than two months or are characterized by marked functional impairment, morbid preoccupation with worthlessness, suicidal ideation, psychotic symptoms, or psychomotor retardation.

Major depressive disorder is characterized by the presence of the majority of these symptoms: a depressed mood most of the day, nearly every day, as indicated by either subjective report (e.g., feels sad or empty) or observation made by others (e.g., appears tearful). In children and adolescents, this may be characterized as an irritable mood. A markedly diminished interest or pleasure in all, or almost all, activities most of the day, nearly every day; significant weight loss when not dieting or weight gain (e.g., a change of more than 5% of body weight in a month), or decrease or increase in appetite nearly every day; insomnia or hypersomnia nearly every day; psychomotor agitation or retardation nearly every day; fatigue or loss of energy nearly every day; feelings of worthlessness or excessive or inappropriate guilt nearly every day; diminished ability to think or concentrate, or indecisiveness, nearly every day; recurrent thoughts of death (not just fear of dying), recurrent suicidal ideation without a specific plan, or a suicide attempt or a specific plan for committing suicide.

Investigation of the biochemical differences between normal individuals and those with major depressive disorder may provide insight into the causes and/or effects of this disorder. In some instances, these differences may constitute biomarkers indicative of the presence and status of the disorder.

Secretogranin II is an acidic secretory protein found in large dense core vesicles of endocrine, neuroendocrine and neuronal tissues. It is a member of a class of proteins termed chromogranins. Secretogranin II is the precursor of the neuropeptide secretoneurin (SN), a 33 amino acid peptide which, in rat, corresponds to amino acids 154-186 of Secretogranin II. Secretoneurin induces dopamine release in the striatum of the rat brain. The secretoneurin 33 amino acid neuropeptide is highly conserved between mammals, reptiles, birds, amphibians and fish. It is specifically expressed in endocrine, neuroendocrine and neuronal tissues. In brain, the pattern of SN expression is widespread and unique, partially overlapping with established neurotransmitters.

The VGF gene encodes a neuropeptide precursor which is expressed in a subset of neurons in the central and peripheral nervous system and in specific populations of endocrine cells found in the adenohypophysis, adrenal medulla, gastrointestinal tract and pancreas. Expression of VGF is upregulated in responsive neurons by neurotrophins. VGF is a recognised nerve growth factor and plays an essential role in the regulation of energy homeostasis. The human VGF protein is 615 amino acids in length; the VGF protein in mouse and rat is 617 amino acids in length. There is about 85% homology between the human and rat VGF proteins. The VGF neuropeptide precursor has a secretory leader (“signal”) sequence of 22 amino acids that promotes translocation into the endoplasmic reticulum. In the VGF neuropeptide precursor and the mature full length VGF peptide cleaved from that precursor, there are numerous short stretches of basic amino acid residues, which are potential target sites for peptidase cleavage resulting in the generation of shorter VGF peptides. VGF peptides have been identified in rat and human; Stark et al, J. Chromatography B, 754, 357-367, 2001, identified three N-terminal fragments of VGF (amino acids 23 to 62, 26 to 62 (N-terminal truncation of peptide 23 to 62) and 23 to 59 (C-terminal truncation of peptide 23 to 62) in human cerebrospinal fluid (CSF) obtained from subjects without known disorders.

VGF peptide biomarkers have been associated with chronic dementia diseases. WO02/082075 describes methods for detecting chronic dementia diseases, in particular Alzheimer's disease, involving detection of various VGF-derived peptides, including VGF 23 to 62 and VGF 26 to 62.

WO2004/082455 and US2004/0142388 also disclose biomarkers for Alzheimer's disease.

WO2006/085121 (not published at the priority date of this case) discloses biomarkers for schizophrenia and bipolar disorder.

### SUMMARY OF THE INVENTION

The present invention relates to peptide biomarkers for major depressive disorders.

The present invention provides an isolated and purified secretogranin II peptide, consisting of the amino acid sequence shown in SEQ ID NO: 1, or a fragment thereof. This peptide, which corresponds to amino acids 529-566 of human Secretogranin II, has been found to be present at a reduced level in subjects with major depressive disorder compared to the level found in normal subjects and is thus useful as a biomarker for major depressive disorder, or predisposition thereto. Accordingly, the present invention provides the use of a secretogranin II peptide, preferably consisting of the amino acid sequence shown in SEQ ID NO: 1, or a fragment thereof, as a biomarker for major depressive disorder, or predisposition thereto. The term Secretogranin II peptide biomarker includes the peptide which corresponds to amino acids 529-566 of human Secretogranin II and fragments of the secretogranin II 529-566 peptide.

The invention further provides a Secretogranin II peptide biomarker for major depressive disorder, or predisposition thereto, preferably consisting of the amino acid sequence shown in SEQ ID NO: 1, or a fragment thereof.

The present invention provides the use of a VGF peptide, preferably consisting of the amino acid sequence shown in SEQ ID NO: 4, or a fragment thereof, as a biomarker for major depressive disorder, or predisposition thereto.

The invention further provides a VGF peptide biomarker for major depressive disorder, or predisposition thereto, preferably consisting of the amino acid sequence shown in SEQ ID NO: 4, or a fragment thereof.

In a further aspect, the invention provides a method of diagnosing or monitoring major depressive disorder, or predisposition thereto, comprising detecting and/or quantifying one or more peptide biomarker selected from the group consisting of the amino acid sequence of SEQ ID NO: 1, a fragment thereof; the amino acid sequence of SEQ ID NO: 4 and a fragment thereof, present in a biological sample from a test subject.

A further aspect of the invention provides ligands, such as naturally occurring or chemically synthesised compounds, capable of specific binding either to the Secretogranin II peptide biomarker of SEQ ID NO: 1 or a fragment thereof, or to the VGF peptide biomarker of SEQ ID NO: 4 or a fragment thereof. A ligand according to the invention may comprise a peptide, an antibody or a fragment thereof, or an aptamer or oligonucleotide, capable of specific binding either to the Secretogranin II peptide biomarker of SEQ ID NO: 1 or a fragment thereof or to the VGF peptide biomarker of SEQ ID NO: 4 or a fragment thereof. The antibody can be a monoclonal antibody or a fragment thereof capable of specific binding either to the Secretogranin II peptide biomarker of SEQ ID NO: 1 or a fragment thereof or to the VGF peptide biomarker of SEQ ID NO: 4 or a fragment thereof. A ligand according to the invention may be labelled with a detectable marker, such as a luminescent, fluorescent or radioactive marker; alternatively or additionally a ligand according to the invention may be labelled with an affinity tag, e.g. a biotin, avidin, streptavidin or his (e.g. hexa-his) tag.

Biosensors according to the invention may comprise a ligand or ligands, as described herein, capable of specific binding either to the Secretogranin II peptide biomarker of SEQ ID NO: 1 or a fragment thereof; or to the VGF peptide biomarker of SEQ ID NO: 4 or a fragment thereof. Such biosensors are useful in detecting and/or quantifying a peptide of the invention.

A biosensor according to the invention may comprise the Secretogranin II peptide biomarker of SEQ ID NO: 1 or a fragment thereof and/or VGF peptide biomarker of SEQ ID NO: 4 or a fragment thereof; or a structural/shape mimic thereof capable of specific binding to an antibody against the Secretogranin II peptide biomarker or a fragment thereof, or capable of specific binding to an antibody against the VGF peptide biomarker or a fragment thereof.

Also provided is an array comprising a ligand as described herein capable of specific binding to the Secretogranin II peptide biomarker of SEQ ID NO: 1 or a fragment thereof and/or a ligand as described herein capable of specific binding to the VGF peptide biomarker of SEQ ID NO: 4 or a fragment thereof; or an array comprising the Secretogranin II peptide biomarker of SEQ ID NO: 1 or a fragment thereof or a structural/shape mimic thereof and/or a VGF peptide biomarker of SEQ ID NO: 4 or fragment thereof or a structural/shape mimic thereof.

Diagnostic or monitoring kits are provided for performing methods of the invention. Such kits will suitably comprise one or more ligand according to the invention, for detection and/or quantification of one or more peptide biomarkers selected from Secretogranin II peptide biomarker of SEQ ID NO:1, a fragment thereof, the VGF peptide biomarker of SEQ ID NO: 4 and a fragment thereof, and/or a biosensor, and/or an array as described herein, optionally together with instructions for use of the kit.

Also provided by the invention is the use of one or more ligands as described herein, which may be naturally occurring or chemically synthesised, and is suitably a peptide, antibody or fragment thereof, aptamer or oligonucleotide, or the use of a biosensor of the invention, or an array of the invention, or a kit of the invention to detect and/or quantify one or more peptide selected from the Secretogranin II peptide biomarker of SEQ ID NO: 1, a fragment thereof; the VGF peptide biomarker of SEQ ID NO: 4 and a fragment thereof. In these uses, the detection and/or quantification can be performed on a biological sample such as from the group consisting of CSF, whole blood, blood serum, plasma, urine, saliva, or other bodily fluid, breath, e.g. as condensed breath, or an extract or purification therefrom, or dilution thereof.

Biomarkers for major depressive disorders are essential targets for discovery of novel targets and drug molecules that retard or halt progression of the disorder. As the level of the peptide biomarker is indicative of disorder and of drug response, the biomarker is useful for identification of novel therapeutic compounds in in vitro and/or in vivo assays. Biomarkers of the invention can be employed in methods for screening for compounds that modulate the activity of a Secretogranin II or VGF peptide biomarker according to the invention; or promote the generation of a Secretogranin II peptide biomarker of SEQ ID NO: 1 or suppress the generation of a VGF peptide biomarker of SEQ ID NO: 4.

Thus, in a further aspect of the invention, there is provided the use of a ligand, as described, which can be a peptide, antibody or fragment thereof or aptamer or oligonucleotide according to the invention; or the use of a biosensor according to the invention, or an array according to the invention; or a kit according to the invention, to identify a substance capable of promoting the generation of a Secretogranin II peptide biomarker and/or of suppressing the generation of a VGF peptide biomarker.

Also there is provided a method of identifying a substance capable of promoting the generation of a Secretogranin II peptide biomarker (preferably consisting of the amino acid sequence of SEQ ID NO: 1, or a fragment thereof) in a subject, comprising administering a test substance to a subject animal and detecting and/or quantifying the level of the Secretogranin II peptide biomarker present in a test sample from the subject.

Also there is provided a method of identifying a substance capable of suppressing the generation of a VGF peptide biomarker (preferably consisting of the amino acid sequence of SEQ ID NO: 4, or a fragment thereof) in a subject, comprising administering a test substance to a subject animal and detecting and/or quantifying the level of the VGF peptide biomarker present in a test sample from the subject.

### SEQUENCE LISTING INFORMATION

SEQ ID NO: 1 is of the human Secretogranin II 529-566 peptide biomarker.

SEQ ID NO: 2 is of the full-length human Secretogranin II protein.

SEQ ID NO: 3 is of the nucleic acid sequence encoding the full-length human Secretogranin II protein.

SEQ ID NO: 4 is of the human VGF 23-62 peptide biomarker.

SEQ ID NO: 5 is of the human VGF precursor protein.

SEQ ID NO: 6 is the human VGF 26-62 peptide amino acid sequence.

### DESCRIPTION OF THE INVENTION

The term “VGF peptide biomarker” includes the mature full-length human VGF peptide generated by cleavage of the signal sequence from the human VGF neuropeptide precursor. Preferred VGF peptide biomarkers are peptides in which the N-terminus is generated by proteolytic cleavage of the putative secretory leader (“signal”) sequence of VGF. A particularly preferred VGF peptide biomarker (SEQ ID NO: 4) is derived from the human VGF protein, the biomarker consists of amino acids 23 to 62 of VGF. This biomarker amino acid sequence is found immediately following the carboxyl terminus of the putative signal peptide in human VGF protein (FIG. 4). The peptide biomarker as shown in SEQ ID NO: 4 is found to be present at elevated levels in individuals with major depressive disorder, it is thus useful as a marker for diagnosing and monitoring major depressive disorder, or predisposition thereto.

The term “biomarker” means a distinctive biological or biologically derived indicator of a process, event, or condition. Peptide biomarkers can be used in methods of diagnosis, e.g. clinical screening, and prognosis assessment and in monitoring the results of therapy, identifying patients most likely to respond to a particular therapeutic treatment, drug screening and development. Biomarkers and uses thereof are valuable for identification of new drug treatments and for discovery of new targets for drug treatment.

The term “major depressive disorder” refers to certain types of depression. The diagnostic category major depressive disorder appears in the Diagnostic and Statistical Manual of Mental Disorders of the American Psychiatric Association. The term is generally not used in countries which use the ICD-10 system, in which the equivalent term is “depressive episode”.

Major Depression, or, more properly, Major Depressive Disorder (MDD), is characterized by a severely depressed mood that persists for at least two weeks. Major Depressive Disorder is specified as either “a single episode” or “recurrent”; periods of depression may occur as discrete events or as recurrent events over the lifespan of a subject. Episodes of major depression may be further divided into mild, major or severe. Where the patient has already had an episode of mania or markedly elevated mood, a diagnosis of bipolar disorder (also called bipolar affective disorder) is usually made instead of MDD; depression without periods of elation or mania is therefore sometimes referred to as unipolar depression because their mood remains on one pole. The diagnosis also usually excludes cases where the symptoms are a normal result of bereavement.

Diagnosticians recognize several possible subtypes of Major Depressive Disorder. ICD-10 does not specify a melancholic subtype, but does distinguish on presence or absence of psychosis.

Depression with Catatonic Features—This subtype can be applied to Major Depressive episodes as well as to manic episodes, though it is rare, and rarer in mania. Catatonia is characterized by motoric immobility evidenced by catalepsy or stupor. This MDD subtype may also manifest excessive, non-prompted motor activity (akathisia), extreme negativism or mutism, and peculiarities in movement, including stereotypical movements, prominent mannerisms, and prominent grimacing. There may also be evidence of echolalia or echopraxia. It is very rarely encountered, and may not be a useful category.

Depression with Melancholic Features—Melancholia is characterized by a loss of pleasure (anhedonia) in most or all activities, a failure of reactivity to pleasurable stimuli, a quality of depressed mood more pronounced than that of grief or loss, a worsening of symptoms in the morning hours, early morning waking, psychomotor retardation, anorexia (excessive weight loss, not to be confused with Anorexia Nervosa), or excessive guilt.

Depression with Atypical Features—Atypicality is characterized by mood reactivity (paradoxical anhedonia) and positivity, significant weight gain or increased appetite, excessive sleep or somnolence (hypersomnia), leaden paralysis, or significant social impairment as a consequence of hypersensitivity to perceived interpersonal rejection. People with this can react with interest or pleasure to some things, unlike most depressed individuals.

Depression with Psychotic Features—Some people with a Major Depressive or Manic episode may experience psychotic features. They may be presented with hallucinations or delusions that are either mood-congruent (content coincident with depressive themes) or non-mood-congruent (content not coincident with depressive themes). It is clinically more common to encounter a delusional system as an adjunct to depression than to encounter hallucinations, whether visual or auditory.

Other categories of depression which are not encompassed by the term major depressive disorder include:

Dysthymia, a long-term, mild depression that lasts for a minimum of two years. There must be persistent depressed mood continuously for at least two years. By definition the symptoms are not as severe as with Major Depression, although those with Dysthymia are vulnerable to co-occurring episodes of Major Depression. This disorder often begins in adolescence and may persist for life. People who are diagnosed with major depressive episodes and dysthymic disorder are diagnosed with double depression. Dysthymic disorder develops first and then one or more major depressive episodes happen later.

Bipolar I Disorder is an episodic illness in which moods may cycle between mania and depression. In the United States, Bipolar Disorder was previously called Manic Depression. This term is no longer favoured by the medical community, however, even though depression plays a much stronger (in terms of disability and potential for suicide) role in the disorder. “Manic Depression” is still often used in the non-medical community.

Bipolar II Disorder is an episodic illness that is defined primarily by depression but evidences episodes of hypomania.

Postpartum Depression or Post-Natal Depression is clinical depression that occurs within two years of childbirth, largely due to physical, mental and emotional exhaustion combined with sleep-deprivation.

Premenstrual dysphoria is a pattern of recurrent depressive symptoms tied to the menstrual cycle. The premenstrual decline in brain serotonin function is strongly correlated with the concomitant worsening of self-rated cardinal mood symptoms.

Furthermore, the term “Major depressive disorder” does not encompass schizophrenic disorders, bipolar disorders and related psychotic disorders such as neuropsychiatric (psychotic depression and other psychotic episodes) and neurodevelopmental disorders (especially Autistic spectrum disorders) which can present with psychotic or other schizophrenia-like symptoms.

Monitoring methods of the invention can be used to monitor onset, progression, stabilisation, amelioration and/or remission.

In methods of diagnosing or monitoring according to the invention, detecting and/or quantifying the peptide biomarker(s) in a biological sample from a test subject may be performed on two or more occasions. Comparisons may be made between the level of biomarker(s) in samples taken on two or more occasions. Assessment of any change in the level of the peptide biomarker(s) in samples taken on two or more occasions may be performed. Modulation of the peptide biomarker level is useful as an indicator of the state of the major depressive disorder or predisposition thereto. A decrease in the level of a secretogranin II peptide biomarker, preferably consisting of the amino acid sequence of SEQ ID NO: 1 or a fragment thereof, over time is indicative of onset or progression, i.e. worsening of this disorder, whereas an increase in the level of the peptide biomarker indicates amelioration or remission of the disorder.

An increase in the level of a VGF peptide biomarker, preferably consisting of the amino acid sequence of SEQ ID NO: 4 or a fragment thereof, over time is indicative of onset or progression, i.e. worsening of this disorder, whereas a decrease in the level of the peptide biomarker indicates amelioration or remission of the disorder.

A method of diagnosis of or monitoring according to the invention may comprise quantifying a secretogranin II peptide biomarker, preferably consisting of the amino acid sequence of SEQ ID NO: 1 or a fragment thereof, and/or quantifying a VGF peptide biomarker, preferably consisting of the amino acid sequence of SEQ ID NO: 4, or a fragment thereof, in a test biological sample from a test subject and comparing the level of the peptide present in said test sample with one or more controls.

The control used in a method of the invention can be one or more control(s) selected from the group consisting of: the level of biomarker peptide found in a normal control sample from a normal subject, a normal biomarker peptide level; a normal biomarker peptide range, the level in a sample from a subject with a major depressive disorder, or a diagnosed predisposition thereto; a major depressive disorder biomarker peptide level, or a major depressive disorder biomarker peptide range.

A preferred method of diagnosing a major depressive disorder, or predisposition thereto, comprises:
(a) quantifying the amount of a secretogranin II peptide biomarker, preferably consisting of SEQ ID NO: 1, or a fragment thereof, in a test biological sample, and/or,
(b) quantifying the amount of a VGF peptide biomarker, preferably consisting of SEQ ID NO: 4, or a fragment thereof, in a test biological sample, and,
(c) comparing the amount of said peptide in said test sample with the amount present in a normal control biological sample from a normal subject.

A lower level of the secretogranin II peptide biomarker in the test sample relative to the level in the normal control is indicative of the presence of a major depressive disorder, or predisposition thereto; an equivalent or higher level of the peptide in the test sample relative to the normal control is indicative of absence of a major depressive disorder and/or absence of a predisposition thereto.

A higher level of the VGF peptide biomarker in the test sample relative to the level in the normal control is indicative of the presence of a major depressive disorder, or predisposition thereto; an equivalent or lower level of the peptide in the test sample relative to the normal control is indicative of absence of a major depressive disorder and/or absence of a predisposition thereto.

The term “diagnosis” as used herein encompasses identification, confirmation, and/or characterisation of a major depressive disorder, or predisposition thereto. By predisposition it is meant that a subject does not currently present with the disorder, but is liable to be affected by the disorder in time. Methods of monitoring and of diagnosis according to the invention are useful to confirm the existence of a disorder, or predisposition thereto; to monitor development of the disorder by assessing onset and progression, or to assess amelioration or regression of the disorder. Methods of monitoring and of diagnosis are also useful in methods for assessment of clinical screening, prognosis, choice of therapy, evaluation of therapeutic benefit, i.e. for drug screening and drug development.

Efficient diagnosis and monitoring methods provide very powerful “patient solutions” with the potential for improved prognosis, by establishing the correct diagnosis, allowing rapid identification of the most appropriate treatment (thus lessening unnecessary exposure to harmful drug side effects), reducing “down-time” and relapse rates.

Also provided is a method of monitoring efficacy of a therapy for a major depressive disorder in a subject having such a disorder, suspected of having such a disorder, or of being predisposed thereto, comprising detecting and/or quantifying a Secretogranin II peptide, preferably consisting of the amino acid sequence of SEQ ID NO: 1, or a fragment thereof, and/or comprising detecting and/or quantifying a VGF peptide, preferably consisting of the amino acid sequence of SEQ ID NO: 4 or a fragment thereof, present in a biological sample from said subject. In monitoring methods, test samples may be taken on two or more occasions. The method may further comprise comparing the level of the biomarker(s) present in the test sample with one or more control(s) and/or with one or more previous test sample(s) taken earlier from the same test subject, e.g. prior to commencement of therapy, and/or from the same test subject at an earlier stage of therapy. The method may comprise detecting a change in the level of the biomarker(s) in test samples taken on different occasions.

The invention provides a method for monitoring efficacy of therapy for major depressive disorder in a subject, comprising:
(a) quantifying the amount of a Secretogranin II peptide biomarker, preferably consisting of the amino acid sequence of SEQ ID NO: 1 or a fragment thereof, in a test biological sample taken from said subject, and/or,
(b) quantifying the amount of a VGF peptide biomarker, preferably consisting of the amino acid sequence of SEQ ID NO: 4 or a fragment thereof, in a test biological sample taken from said subject, and
(c) comparing the amount of said peptide(s) in said test sample with the amount present in one or more control(s) and/or one or more previous test sample(s) taken at an earlier time from said same test subject.

An increase in the level of the Secretogranin II peptide biomarker in the test sample relative to the level in a previous test sample taken earlier from the same test subject is indicative of a beneficial effect, e.g. stabilisation or improvement, of said therapy on the disorder, suspected disorder or predisposition thereto.

A decrease in the level of the VGF peptide biomarker in the test sample relative to the level in a previous test sample taken earlier from the same test subject is indicative of a beneficial effect, e.g. stabilisation or improvement, of said therapy on the disorder, suspected disorder or predisposition thereto.

Methods for monitoring efficacy of a therapy can be used to monitor the therapeutic effectiveness of existing therapies and new therapies in human subjects and in non-human animals (e.g. in animal models). These monitoring methods can be incorporated into screens for new drug substances and combinations of substances.

Suitably, the time elapsed between taking samples from a subject undergoing diagnosis or monitoring will be 3 days, 5 days, a week, two weeks, a month, 2 months, 3 months, 6 or 12 months. Samples may be taken prior to and/or during and/or following an anti-depressant therapy. Samples can be taken at intervals over the remaining life, or a part thereof, of a subject.

The term “detecting” as used herein means confirming the presence of the peptide biomarker present in the sample. Quantifying the amount of the biomarker present in a sample may include determining the concentration of the peptide biomarker present in the sample. Detecting and/or quantifying may be performed directly on the sample, or indirectly on an extract therefrom, or on a dilution thereof.

In alternative aspects of the invention, the presence of the peptide biomarker is assessed by detecting and/or quantifying antibody or fragments thereof capable of specific binding to the biomarker that are generated by the subject's body in response to the peptide and thus are present in a biological sample from a subject having major depressive disorder or a predisposition thereto.

Detecting and/or quantifying can be performed by any method suitable to identify the presence and/or amount of a specific protein in a biological sample from a patient or a purification or extract of a biological sample or a dilution thereof. In methods of the invention, quantifying may be performed by measuring the concentration of the peptide biomarker in the sample or samples. Biological samples that may be tested in a method of the invention include cerebrospinal fluid (CSF), whole blood, blood serum, plasma, urine, saliva, or other bodily fluid (stool, tear fluid, synovial fluid, sputum), breath, e.g. as condensed breath, or an extract or purification therefrom, or dilution thereof. Biological samples also include tissue homogenates, tissue sections and biopsy specimens from a live subject, or taken post-mortem. The samples can be prepared, for example where appropriate diluted or concentrated, and stored in the usual manner.

Detection and/or quantification of Secretogranin II or VGF peptide biomarkers may be performed by detection of the peptide biomarker or of a fragment thereof, e.g. a fragment with C-terminal truncation, or with N-terminal truncation. Fragments are suitably greater than 4 amino acids in length, preferably 5, 6, 7, 8, 9, 10, 11, 12, 13, 14, 15, 16, 17, 18, 19, or 20 amino acids in length.

The biomarker may be directly detected, e.g. by SELDI or MALDI-TOF. Alternatively, the biomarker may be detected directly or indirectly via interaction with a ligand or ligands such as an antibody or a biomarker-binding fragment thereof, or other peptide, or ligand, e.g. aptamer, or oligonucleotide, capable of specifically binding the biomarker. The ligand may possess a detectable label, such as a luminescent, fluorescent or radioactive label, and/or an affinity tag.

For example, detecting and/or quantifying can be performed by one or more method(s) selected from the group consisting of: SELDI(-TOF), MALDI(-TOF), a 1-D gel-based analysis, a 2-D gel-based analysis, Mass spec (MS), reverse phase (RP) LC, size permeation (gel filtration), ion exchange, affinity, HPLC, HPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods of diagnosing or monitoring according to the invention may comprise analysing a sample of cerebrospinal fluid (CSF) by SELDI TOF or MALDI TOF to detect the presence or level of the peptide biomarker of SEQ ID NO: 1 and/or SEQ ID NO: 4. These methods are also suitable for clinical screening, prognosis, monitoring the results of therapy, identifying patients most likely to respond to a particular therapeutic treatment, for drug screening and development, and identification of new targets for drug treatment.

Detecting and/or quantifying the peptide biomarkers may be performed using an immunological method, involving an antibody, or a fragment thereof capable of specific binding to the Secretogranin II peptide biomarker, e.g. to a peptide consisting of the amino acid sequence shown in SEQ ID NO: 1 or a fragment thereof; or capable of specific binding to the VGF peptide biomarker, e.g. to a peptide consisting of the amino acid sequence shown in SEQ ID NO: 4 or a fragment thereof. Suitable immunological methods include sandwich immunoassays, such as sandwich ELISA, in which the detection of the peptide biomarkers is performed using two antibodies which recognize different epitopes on a peptide biomarker; radioimmunoassays (RIA), direct, indirect or competitive enzyme linked immunosorbent assays (ELISA), enzyme immuno assays (EIA), Fluorescence immunoassays (FIA), western blotting, immunoprecipitation and any particle-based immunoassay (e.g. using gold, silver, or latex particles, magnetic particles, or Q-dots). Immunological methods may be performed, for example, in microtitre plate or strip format.

Immunological methods in accordance with the invention may be based, for example, on any of the following methods.

Immunoprecipitation is the simplest immunoassay method; this measures the quantity of precipitate, which forms after the reagent antibody has incubated with the sample and reacted with the target antigen present therein (in this instance the target antigen is a peptide biomarker of SEQ ID NO: 1, a fragment thereof, SEQ ID NO: 4 or a fragment thereof) to form an insoluble aggregate. Immunoprecipitation reactions may be qualitative or quantitative.

In particle immunoassays, several antibodies are linked to the particle, and the particle is able to bind many antigen molecules simultaneously. This greatly accelerates the speed of the visible reaction. This allows rapid and sensitive detection of the biomarker.

In immunonephelometry, the interaction of an antibody and target antigen on the biomarker results in the formation of immune complexes that are too small to precipitate. However, these complexes will scatter incident light and this can be measured using a nephelometer. The antigen, i.e. biomarker, concentration can be determined within minutes of the reaction.

Radioimmunoassay (RIA) methods employ radioactive isotopes such as 1¹²⁵ to label either the antigen or antibody. The isotope used emits gamma rays, which are usually measured following removal of unbound (free) radiolabel. The major advantages of RIA, compared with other immunoassays, are higher sensitivity, easy signal detection, and well-established, rapid assays. The major disadvantages are the health and safety risks posed by the use of radiation and the time and expense associated with maintaining a licensed radiation safety and disposal program. For this reason, RIA has been largely replaced in routine clinical laboratory practice by enzyme immunoassays.

Enzyme (EIA) immunoassays were developed as an alternative to radioimmunoassays (RIA). These methods use an enzyme to label either the antibody or target antigen. The sensitivity of EIA approaches that for RIA, without the danger posed by radioactive isotopes. One of the most widely used EIA methods for detection is the enzyme-linked immunosorbent assay (ELISA). ELISA methods may use two antibodies one of which is specific for the target antigen and the other of which is coupled to an enzyme, addition of the substrate for the enzyme results in production of a chemoluminescent or fluorescent signal.

Fluorescent immunoassay (FIA) refers to immunoassays which utilize a fluorescent label or an enzyme label which acts on the substrate to form a fluorescent product. Fluorescent measurements are inherently more sensitive than colorimetric (spectrophotometric) measurements. Therefore, FIA methods have greater analytical sensitivity than EIA methods, which employ absorbance (optical density) measurement.

Chemiluminescent immunoassays utilize a chemiluminescent label, which produces light when excited by chemical energy; the emissions are measured using a light detector.

Immunological methods according to the invention can thus be performed using well-known methods. Any direct (e.g., using a sensor chip) or indirect procedure may be used in the detection of peptide biomarkers of the invention.

The Biotin-Avidin or Biotin-Streptavidin systems are generic labelling systems that can be adapted for use in immunological methods of the invention. One binding partner (hapten, antigen, ligand, aptamer, antibody, enzyme etc) is labelled with biotin and the other partner (surface, e.g. well, bead, sensor etc) is labelled with avidin or streptavidin. This is conventional technology for immunoassays, gene probe assays and (bio)sensors, but is an indirect immobilisation route rather than a direct one. For example a biotinylated ligand (e.g. antibody or aptamer) specific for a peptide biomarker of the invention may be immobilised on an avidin or streptavidin surface, the immobilised ligand may then be exposed to a sample containing or suspected of containing the peptide biomarker in order to detect and/or quantify a peptide biomarker of the invention. Detection and/or quantification of the immobilised antigen may then be performed by an immunological method as described herein.

The term “antibody” as used herein includes, but is not limited to: polyclonal, monoclonal, bispecific, humanised or chimeric antibodies, single chain antibodies, Fab fragments and F(ab′)₂ fragments, fragments produced by a Fab expression library, anti-idiotypic (anti-Id) antibodies and epitope-binding fragments of any of the above. The term “antibody” as used herein also refers to immunoglobulin molecules and immunologically-active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule.

The identification of key biomarkers specific to a disease is central to integration of diagnostic procedures and therapeutic regimes. Using predictive biomarkers appropriate diagnostic tools such as biosensors can be developed, accordingly, in methods and uses of the invention, detecting and quantifying can be performed using a biosensor, microanalytical system, microengineered system, microseparation system, immunochromatography system or other suitable analytical devices. The biosensor may incorporate an immunological method for detection of the biomarker(s), electrical, thermal, magnetic, optical (e.g. hologram) or acoustic technologies. Using such biosensors, it is possible to detect the target biomarker(s) at the anticipated concentrations found in biological samples.

The biomarker(s) of the invention can be detected using a biosensor incorporating technologies based on “smart” holograms, or high frequency acoustic systems, such systems are particularly amenable to “bar code” or array configurations.

In smart hologram sensors (Smart Holograms Ltd, Cambridge, UK), a holographic image is stored in a thin polymer film that is sensitised to react specifically with the biomarker. On exposure, the biomarker reacts with the polymer leading to an alteration in the image displayed by the hologram. The test result read-out can be a change in the optical brightness, image, colour and/or position of the image. For qualitative and semi-quantitative applications, a sensor hologram can be read by eye, thus removing the need for detection equipment. A simple colour sensor can be used to read the signal when quantitative measurements are required. Opacity or colour of the sample does not interfere with operation of the sensor. The format of the sensor allows multiplexing for simultaneous detection of several substances. Reversible and irreversible sensors can be designed to meet different requirements, and continuous monitoring of a particular biomarker of interest is feasible.

Suitably, biosensors for detection of one or more biomarkers of the invention combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace.

Biosensors to detect one or more biomarkers of the invention include acoustic, plasmon resonance, holographic and microengineered sensors. Imprinted recognition elements, thin film transistor technology, magnetic acoustic resonator devices and other novel acousto-electrical systems may be employed in biosensors for detection of the one or more biomarkers of the invention.

Methods involving detection and/or quantification of one or more peptide biomarkers of the invention can be performed on bench-top instruments, or can be incorporated onto disposable, diagnostic or monitoring platforms that can be used in a non-laboratory environment, e.g. in the physician's office or at the patient's bedside. Suitable biosensors for performing methods of the invention include “credit” cards with optical or acoustic readers. Biosensors can be configured to allow the data collected to be electronically transmitted to the physician for interpretation and thus can form the basis for e-neuromedicine.

In methods, uses and biosensors of the invention in which the amount of the Secretogranin II biomarker peptide of SEQ ID NO: 1 or a fragment thereof present in a test sample from a test subject is measured, detection of a lower level of the biomarker peptide in the test sample compared to the level found in a normal control sample from a normal individual is indicative of a major depressive disorder, or a predisposition thereto in the test subject. For example, the level of peptide of SEQ ID NO: 1 detected in a sample from a test subject with a major depressive disorder or predisposition thereto will generally be 40% lower, than the amount of the peptide found in a normal control sample. Expressed as a ratio, a lower level of peptide of SEQ ID NO: 1 indicative of a major depressive disorder or a predisposition thereto exists when the ratio of the amount of peptide of SEQ ID NO: 1 in a test sample compared to a normal control is below 0.75:1.

In methods, uses and biosensors of the invention in which the amount of the VGF biomarker peptide of SEQ ID NO: 4 or a fragment thereof present in a test sample from a test subject is measured, detection of a higher level of the biomarker peptide in the test sample compared to the level found in a normal control sample from a normal individual is indicative of a major depressive disorder, or a predisposition thereto in the test subject.

For example, the level of a VGF peptide of SEQ ID NO: 4 detected in a sample from a test subject with a major depressive disorder or predisposition thereto will generally be in the range of above 1.3-fold, e.g. 1.5-fold higher than the amount of the peptide found in a normal control sample. Expressed as a ratio, a higher level of peptide of SEQ ID NO: 4 indicative of a major depressive disorder or a predisposition thereto exists when the ratio of the amount of peptide of SEQ ID NO: 4 in a test sample compared to a normal control is above 1.3-fold higher (but below 2.5-fold higher), e.g. about 1.5:1.

The VGF peptide biomarker of SEQ ID NO: 4 has been found at a very elevated level in drug naïve subjects with first onset psychosis associated with schizophrenia. In those subjects, the level of the VGF peptide of SEQ ID NO: 4 detected was approximately 2.5-fold higher than the amount of the peptide found in a normal control sample. By assessing the level of the VGF peptide of SEQ ID NO: 4 present in a test sample it is possible to detect, and to distinguish between MDD and first onset psychosis associated with schizophrenia.

Any suitable animal may be used as a subject non-human animal, for example a non-human primate, horse, cow, pig, goat, sheep, dog, cat, fish, rodent, e.g. guinea pig, rat or mouse; insect (e.g. Drosophila), amphibian (e.g. Xenopus) or C. elegans.

The test substance can be a known chemical or pharmaceutical substance, such as, but not limited to, an anti-depressive disorder therapeutic; or the test substance can be novel synthetic or natural chemical entity, or a combination of two or more of the aforesaid substances.

There is provided a method of identifying a substance capable of promoting the generation of a Secretogranin II peptide biomarker, preferably consisting of the amino acid sequence of SEQ ID NO: 1, or a fragment thereof, in a subject, comprising exposing a test cell to a test substance and monitoring the level of the Secretogranin II peptide biomarker within said test cell, or secreted by said test cell.

There is provided a method of identifying a substance capable of suppressing the generation of a VGF peptide biomarker, preferably consisting of the amino acid sequence of SEQ ID NO: 4, or a fragment thereof, in a subject, comprising exposing a test cell to a test substance and monitoring the level of the VGF peptide biomarker within said test cell, or secreted by said test cell.

The test cell could be prokaryotic, however it is preferred that a eukaryotic cell be employed in cell-based testing methods. Suitably, the eukaryotic cell is a yeast cell, insect cell, Drosophila cell, amphibian cell (e.g. from Xenopus), C. elegans cell or is a cell of human, non-human primate, equine, bovine, porcine, caprine, ovine, canine, feline, piscine, rodent or murine origin.

In methods for identifying substances of potential therapeutic use, non-human animals or cells can be used that are capable of expressing one or more polypeptide selected from the group consisting of human Secretogranin II polypeptides and proteolytic enzymes, preferably human proteolytic enzymes capable of cleaving a human Secretogranin II polypeptide, human VGF polypeptides and proteolytic enzymes, preferably human proteolytic enzymes capable of cleaving a human VGF polypeptide. When non-human animals or non-human animal cells are used, methods and uses may involve detecting the Secretogranin II or VGF peptide biomarker which is the animal's equivalent to the human Secretogranin II or VGF biomarker, i.e. the animal homolog of the human Secretogranin peptide II of SEQ ID NO: 1 or the animal homolog of the human VGF peptide of SEQ ID NO: 4.

Screening methods also encompass a method of identifying a ligand capable of binding to a Secretogranin II or VGF peptide biomarker according to the invention, comprising incubating a test substance in the presence of the peptide biomarker in conditions appropriate for binding, and detecting and/or quantifying binding of the peptide to said test substance.

Where the Secretogranin II peptide biomarker is a peptide consisting of the sequence of SEQ ID NO: 1 (human Secretogranin II amino acids 529-566) or a fragment thereof, specific binding is indicated if the test substance binds to the Secretogranin II peptide biomarker (human Secretogranin II amino acids 529-566 or a fragment thereof) but does not bind to full length Secretogranin II, Secretoneurin (SgII 154-186) or Secretogranin II-derived peptide (SgII 187-252).

Secretogranin II (SgII) possesses nine pairs of basic residues in mammals, proteolytic cleavage is known to occur at paired dibasic sequences, suggesting that the SGII protein may be processed to more than 10 peptides. Two of them, secretoneurin (SgII 154-186) and secretogranin II-derived peptide (SgII187-252) have been confirmed as neuropeptides in human.

Where the VGF peptide biomarker is a peptide consisting of the sequence of SEQ ID NO: 4 (human VGF amino acids 23 to 62) or a fragment thereof, specific binding is indicated if the test substance does not bind to mature full length human VGF protein or to a protein consisting of amino acids 26 to 62 of human VGF (N-terminal 3 peptide truncated sequence).

High-throughput screening technologies based on the biomarker, uses and methods of the invention, e.g. configured in an array format, are suitable to monitor biomarker signatures for the identification of potentially useful therapeutic compounds, e.g. ligands such as natural compounds, synthetic chemical compounds (e.g. from combinatorial libraries), peptides, monoclonal or polyclonal antibodies or fragments thereof, which may be capable of binding the biomarker.

Methods of the invention can be performed in array format, e.g. on a chip, or as a multiwell array. Methods can be adapted into platforms for single tests, or multiple identical or multiple non-identical tests, and can be performed in high throughput format. Methods of the invention may comprise performing one or more additional, different tests to confirm or exclude diagnosis, and/or to further characterise a condition.

The invention further provides a substance, e.g. a ligand, identified or identifiable by an identification or screening method or use of the invention. Such substances may be capable of promoting, directly or indirectly, the activity of a Secretogranin II peptide biomarker, or of enhancing generation of the Secretogranin II peptide biomarker. Such substances may be capable of inhibiting, directly or indirectly, the activity of a VGF peptide biomarker, or of suppressing generation of the VGF peptide biomarker. The term “substances” includes substances that do not directly bind the peptide biomarker and directly modulate a function, but instead indirectly modulate a function of the peptide biomarker. Ligands are also included in the term substances; ligands of the invention (e.g. a natural or synthetic chemical compound, peptide, aptamer, oligonucleotide, antibody or antibody fragment) are capable of binding, preferably specific binding, to a Secretogranin II peptide, preferably of SEQ ID NO: 1, or a fragment thereof; or are capable of binding, preferably specific binding, to a VGF peptide biomarker, preferably of SEQ ID NO: 4 or a fragment thereof.

The invention further provides the use of a substance according to the invention in the treatment of a major depressive disorder, or predisposition thereto.

Also provided is the use of a substance according to the invention as a medicament.

Yet further provided is the use of a substance according to the invention in the manufacture of a medicament for the treatment of a major depressive disorder, or predisposition thereto.

A kit for diagnosing or monitoring a major depressive disorder, or predisposition thereto is provided. Suitably a kit according to the invention may contain one or more components selected from the group: a ligand specific for a Secretogranin II peptide biomarker, a Secretogranin II peptide biomarker or a structural/shape mimic of a Secretogranin II peptide biomarker; a ligand specific for a VGF peptide biomarker, a VGF peptide biomarker or a structural/shape mimic of a VGF peptide biomarker, one or more controls, one or more reagents and one or more consumables; optionally together with instructions for use of the kit.

The invention yet further provides an isolated and purified peptide consisting of an amino acid sequence of SEQ ID NO: 1 or a fragment thereof of at least 4 amino acids. Also provided is an isolated and purified nucleic acid encoding the amino acid sequence of SEQ ID NO: 1 or a fragment thereof of at least 4 amino acids and an isolated and purified nucleic acid sequence capable of hybridising specifically thereto, preferably complementary thereto.

A nucleic acid capable of hybridising specifically is a nucleic acid capable of hybridising to the target nucleic acid under conditions of moderate to high stringency. Stringency of hybridisation reactions is readily determinable by one of ordinary skill in the art, and generally is an empirical calculation dependent upon sequence length, washing temperature, and salt concentration. In general, longer sequences require higher temperatures for proper annealing, while shorter sequences need lower temperatures. Hybridisation generally depends on the ability of denatured nucleic acid to reanneal when complementary strands are present in an environment below their melting temperature. The higher the degree of complementarity between the sequences, the higher the relative temperature which can be used. As a result, it follows that higher relative temperatures would tend to make the reaction conditions more stringent, while lower temperatures less so. For additional details and explanation of stringency of hybridisation reactions, see Ausubel et al., Current Protocols in Molecular Biology, Wiley Interscience Publishers, (1995).

As defined herein “high stringency” conditions are those that: (1) employ low ionic strength and high temperature for washing, for example 0.015 M sodium chloride/0.0015 M sodium citrate/0.1% sodium dodecyl sulfate at 50° C.; (2) employ during hybridisation a denaturing agent, such as formamide, for example, 50% (v/v) formamide with 0.1% bovine serum albumin/0.1% FicoII/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 mg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderate stringency” conditions may be identified as described by Sambrook et al., Molecular Cloning: A Laboratory Manual, New York: Cold Spring Harbor Press, 1989, and include the use of washing solution and hybridisation conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37° C. to 50° C. The skilled artisan will recognize how to adjust the temperature, ionic strength, etc. as necessary to accommodate factors such as sequence length and the like.

The identification of biomarkers for major depressive disorder permits integration of diagnostic procedures and therapeutic regimes. Currently there are significant delays in determining effective treatment and hitherto it has not been possible to perform rapid assessment of drug response. Traditionally, many anti-depressant therapies have required treatment trials lasting weeks to months for a given therapeutic approach. Detection of a peptide biomarker of the invention can be used to screen subjects prior to their participation in clinical trials. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized brain therapies, as assessment of response can be used to fine-tune dosage, minimise the number of prescribed medications, reduce the delay in attaining effective therapy and avoid adverse drug reactions. Thus by monitoring a biomarker of the invention, patient care can be tailored precisely to match the needs determined by the disorder and the pharmacogenomic profile of the patient, the biomarker can thus be used to titrate the optimal dose, predict a positive therapeutic response and identify those patients at high risk of severe side effects.

Biomarker-based tests provide a first line assessment of ‘new’ patients, and provide objective measures for accurate and rapid diagnosis, in a time frame and with precision, not achievable using the current subjective measures.

Furthermore, diagnostic biomarker tests are useful to identify family members or patients at high risk of developing major depressive disorder. This permits initiation of appropriate therapy, or preventive measures, e.g. managing risk factors. These approaches are recognised to improve outcome and may prevent overt onset of the disorder.

Biomarker monitoring methods, biosensors and kits are also vital as patient monitoring tools, to enable the physician to determine whether relapse is due to worsening of the disorder, poor patient compliance or substance abuse. If pharmacological treatment is assessed to be inadequate, then therapy can be reinstated or increased; a change in therapy can be given if appropriate. As the biomarkers are sensitive to the state of the disorder, they provide an indication of the impact of drug therapy or of substance abuse.

The following Example illustrates the invention.

### Example

### Materials

Age-matched CSF samples were obtained from healthy individuals (n=40) and patients with depression (MDD) (n=16), OCD (n=5) and Alzheimer's disease (n=10). All chemicals were obtained from Sigma. Protein chips and matrices were obtained from Ciphergen (Guildford, UK).

### Preparation of CSF Samples for SELDI Analysis

Aliquots of 5 μl of the CSF samples were processed on strong anion-exchange (Q10) chips according to the manufacturer's protocols (Ciphergen Biosystems). Briefly, the array spots were preactivated twice with binding buffer (100 mM Tris-HCl, pH 9.0) at room temperature for 10 minutes on a shaker (frequency=5 Hz). Then, 50 μl of binding buffer was added into each protein spot prior to the addition of 5 μl CSF samples. The protein chips were incubated on the shaker for 30 minutes at room temperature. The chips were washed twice with binding buffer and once with HPLC grade H₂O, and then air-dried. The chips were then sequentially treated twice with 1 μl of a 100% saturated sinapinic acid (3,5-dimethoxy-4-hydroxycinnamic acid) in 50% acetonitrile and 0.5% trifluoroacetic acid. The chips were analyzed with the Ciphergen ProteinChip™ Reader (model PBSII). Each sample was analyzed twice to confirm reproducibility in identifying the differentially expressed proteins.

### Ciphergen ProteinChip™ SELDI-TOF-MS Analysis

The arrays were analyzed with the Ciphergen ProteinChip Reader (model PBSII). The mass spectra of proteins were generated by using an average of 65 laser shots at a laser intensity of 230-280 arbitrary units. For data acquisition of low molecular weight proteins, the detection size range was between 3 and 20 kDa, with a maximum size of 25 kDa. The laser was focused at 10 kDa. The detector sensitivity was set at 8, and the laser intensity was set at 190. For the high molecular weight proteins, the detection size range was between 20 and 150 kDa, with a maximum size of 250 kDa. The laser was focused at 85 kDa. The detector sensitivity was set at 9, and the laser intensity was set at 260 for the 1:4 dilution and 280. The mass-to-charge ratio (m/z) of each of the proteins captured on the array surface was determined according to externally calibrated standards (Ciphergen Biosystems): bovine insulin (5,733.6 Da), human ubiquitin (8,564.8 Da), bovine cytochrome c (12,230.9 Da), bovine superoxide dismutase (15,591.4 Da), bovine α-lactoglobulin A (18,363.3 Da), horseradish peroxidase (43,240 Da), BSA (66,410 Da), and chicken conalbumin (77,490 Da).

### LC-MS-MS Analysis of CSF Peptides

Proteins were removed from a 50 μl sample of CSF using a Nanosep™ (Pall Corporation) centrifugal ultrafiltration device with a 10 kDa nominal molecular weight cut-off. An aliquot (5 μl) of the filtrate was desalted by solid-phase microextraction on a C18 ZipTip™ (Waters) and the peptides eluted with 0.1% formic acid/50% aqueous acetonitrile (1 μl) directly into a nanospray tip (Protana Engineering). The nanospray tip was inserted into a nanoelectrospray ion source (Protana Engineering) attached to a quadupole-time-of-flight mass spectrometer (Qstar Pulsar i, Applied Biosystems-MDS Sciex) and full scan TOF spectra were acquired for 5-10 minutes over the m/z range 350-1500 atomic mass units. MS/MS spectra were acquired over the m/z range 50-1700 atomic mass units until sufficient signal:noise was attained. The collision energy was optimized during data acquisition to give the widest range of fragment ions.

MS/MS data were manually interpreted to extract “sequence tags” which were used with BioAnalyst™ software (Applied Biosystems) to search the NCBI NRDB database. The search results were confirmed by further manual interpretation of the MS/MS data.

### Statistical Analysis

The data were analyzed with PROTEINCHIP™ data analysis software version 3.0 (Ciphergen Biosystems). For each comparison, the raw intensity data were normalized by using the total ion current of all profiles in the groups compared. The peak intensities were normalized to the total ion current of m/z between 3,000 and 25,000 Da for the low molecular weight range and between 4,000 and 250,000 Da for the high molecular weight range. The Biomarker Wizard application (nonparametric calculations; Ciphergen Biosystems) was used to compile all spectra and autodetect quantified mass peaks. Peak labeling was completed by using second-pass peak selection with 0.2% of the mass window, and estimated peaks were added. Sample statistics were performed on groups of profiles (normal vs. subjects with disorder). Protein differences (fold changes) were calculated among the various groups.

More particularly, CSF proteomic profiles of patients with depression 5 μl CSF samples from 16 depression patients, together with 40 healthy volunteers were analyzed with SELDI-MS using Q10 anion exchanger protein chips at pH 9 (50 mM Tris-HCl). The spectra were processed using Ciphergen Express software and the peaks with an S/N>5 were output and further analysed using SIMCA (Umetrics, Sweden). The Scores plot showed a partial separation between controls and depression patients. The loading plot indicated VGF23-62 and a secretogranin II (529-566) peptide to be the key protein/peptide peaks, contributing the most towards the group separation.

A peptide of 3.96 kDa (or 3.95 kDa from ESI-MS/MS spectrum) was mapped to amino acids 23 to 62 (SEQ ID NO: 4) of the native VGF protein, immediately next to a predicted secretary signal peptide (using InterProScan: European Bioinformatics Institute: www.ebi.ac.uk/cgibin/iprscan). This 3.96 kDa peptide has the amino acid sequence shown in SEQ ID NO: 4.

Sequence alignment of the 3.96 kDa peptide and the 3.69 kDa peptide was conducted. De novo sequencing using ES/MS-MS showed that the 3.69 kDa peptide in the CSF is a three amino acid (at the N-terminus) shorter form of the 3.96 kDa peptide (SEQ ID NO: 6), which is not differentially expressed in CSF (p=0.87) from healthy volunteers and patients with depression.

Analysis of CSF Samples from Patients with Depression, OCD and Alzheimer's Disease

CSF samples from subjects with depression were most prominently characterized by a distinct decrease of a secretogranin II (529-566) peptide. No difference was found between controls and OCD patients. Additionally, no significant difference between control and Alzheimer's disease CSF samples was found. CSF from subjects with depression showed an increase in the VGF23-62 peptide expression; the peptide was found at levels approximately 50% higher than in CSF from normal healthy controls. This peptide has also been identified to be upregulated significantly in first onset, drug-naïve schizophrenia patients; in this group, the level of VGF peptide was significantly higher than in the depression patients.

1-25. (canceled)

26. A method for determining the existence or progression of a depressive disorder, or a predisposition thereto, in a subject, comprising:
(a) obtaining a biological sample from said subject; and
(b) quantifying the level in said biological sample of each of a Secretogranin II peptide and a VGF peptide;
(c) comparing said level with a reference level for each respective peptide;
wherein a decrease in the level of the Secretogranin II peptide, together with an increase in the level of VGF peptide, compared to said respective reference level, is indicative of the existence or progression of said depressive disorder, or predisposition thereto, in said subject.

27. The method of claim 26, wherein said respective reference level is from a non-depressed subject.

28. The method of claim 26, wherein said respective reference level is from the same subject at a different time during therapy for a depressive disorder, and the difference between said level and the respective reference level is indicative of the progress of said therapy for a depressive disorder.

29. The method of claim 27, wherein said level of a VGF peptide in said subject is at least 30% greater than the respective reference level.

30. The method of claim 27, wherein said level of a VGF peptide in said subject is approximately 50% greater than the respective reference level.

31. The method of claim 27, wherein said level of Secretogranin II peptide in said subject is at least 25% less than the respective reference level.

32. The method of claim 26, wherein said Secretogranin II peptide has the amino acid sequence of SEQ ID NO:1.

33. The method of claim 26, wherein said VGF peptide has the amino acid sequence of SEQ ID NO:4.

34. The method of claim 26, wherein said VGF peptide does not include the sequence of SEQ ID NO:6.

35. The method of claim 26, wherein said depressive disorder or predisposition thereto is major depression.

36. The method of claim 26, wherein said biological sample comprises cerebrospinal fluid, whole blood, blood serum, plasma, saliva, urine or other bodily fluid, or breath, condensed breath, or an extract or purification therefrom, or dilution thereof

37. The method of claim 26, wherein said quantifying is performed by a method selected from the group consisting of: SELDI(-TOF), MALDI(-TOF), a 1-D gel-based analysis, a 2-D gel-based analysis, Mass spec (MS), reverse phase (RP) LC, size permeation (gel filtration), ion exchange, affinity, HPLC, HPLC, an LC or LC-MS-based technique, and an immunological method.

38. The method according to claim 37, wherein said quantifying is performed using a biosensor or a microanalytical, microengineered, microseparation or immunochromatography system.

39. The method of claim 37, wherein said immunological method is selected from the group consisting of: an enzyme linked immunosorbent assay (ELISA), a radioimmunoassay (RIA), a fluorescent immunoassay (FIA), a western blot, immunoprecipitation, and a particle-based immunoassay.

40. A method for determining the existence or progression of a depressive disorder, or a predisposition thereto, in a subject, comprising:
(a) obtaining a biological sample from said subject; and
(b) quantifying the level in said biological sample of each of a Secretogranin II peptide having the sequence of SEQ ID NO:1, and a VGF peptide having the amino acid sequence of SEQ ID NO: 4
(c) comparing said level with a reference level for each respective peptide;
wherein a decrease in level of the Secretogranin II peptide of at least 25%, together with an increase in the level of VGF peptide of at least 30%, compared to said respective reference level, is indicative of the existence or progression of said depressive disorder, or predisposition thereto, in said subject.

41. A kit for the detection of a depressive disorder, its progression, or a predisposition thereto, comprising:
(a) an antibody having selective binding affinity for a peptide consisting of the amino acid sequence of SEQ ID NO: 1; and an antibody having selective binding affinity for a peptide consisting of the amino acid sequence of SEQ ID NO: 4, and
(b) a detectable label.

42. The kit of claim 41, wherein said detectable label comprises a luminescent, fluorescent or radioactive marker, or an affinity tag.


SEQUENCE LISTING (6 sequences)

SEQ ID NO: 1 - protein, 38 aa, Homo sapiens
GQGSSEDDLQEEEQIEQAIKEHLNQGSSQETDKLAPVS

SEQ ID NO: 2 - protein, 617 aa, Homo sapiens
MAEAKTHWLGAALSLIPLIFLISGAEAASFQRNQLLQKEPDLRLENVQKFPSPEMIRALEYIENLRQQAHKEESSPDYNPYQGVSVPLQQKENGDESHLPERDSLSEEDWMRIILEALRQAENEPQSAPKENKPYALNSEKNFPMDMSDDYETQQWPERKLKHMQFPPMYEENSRDNPFKRTNEIVEEQYTPQSLATLESVFQELGKLTGPNNQKRERMDEEQKLYTDDEDDIYKANNIAYEDVVGGEDWNPVEEKIESQTQEEVRDSKENIEKNEQINDEMKRSGQLGIQEEDLRKESKDQLSDDVSKVIAYLKRLVNAAGSGRLQNGQNGERATRLFEKPLDSQSIYQLIEISRNLQIPPEDLIEMLKTGEKPNGSVEPERELDLPVDLDDISEADLDHPDLFQNRMLSKSGYPKTPGRAGTEALPDGLSVEDILNLLGMESAANQKTSYFPNPYNQEKVLPRLPYGAGRSRSNQLPKAAWIPHVENRQMAYENLNDKDQELGEYLARMLVKYPEIINSNQVKRVPGQGSSEDDLQEEEQIEQAIKEHLNQGSSQETDKLAPVSKRFPVGPPKNDDTPNRQYWDEDLLMKVLEYLNQEKAEKGREHIAKRAMENM

SEQ ID NO: 3 - DNA, 2490 bp, Homo sapiens
ggcgcagcggctccctataagcagaggagctgtccgtgtgctgaaacggcccgagaagctcgcccggagaacggggaggaatatgctgtggagctcctctgccatataaacaaaaagaggaaatctttcaaacatggctgaagcaaagacccactggcttggagcagccctgtctcttatccctttaattttcctcatctctggggctgaagcagcttcatttcagagaaaccagctgcttcagaaagaaccagacctcaggttggaaaatgtccaaaagtttcccagtcctgaaatgatcagggctttggagtacatagaaaacctccgacaacaagctcataaggaagaaagcagcccagattataatccctaccaaggtgtctctgtcccccttcagcaaaaagaaaatggcgatgaaagccacttgcccgagagggattcactgagtgaagaagactggatgagaataatactcgaagctttgagacaggctgaaaatgagcctcagtctgcaccaaaagaaaataagccctatgccttgaattcagaaaagaactttccaatggacatgagtgatgattatgagacacagcagtggccagaaagaaagcttaagcacatgcaattccctcctatgtatgaagagaattccagggataacccctttaaacgcacaaatgaaatagtggaggaacaatatactcctcaaagccttgctacattggaatctgtcttccaagagctggggaaactgacaggaccaaacaaccagaaacgtgagaggatggatgaggagcaaaaactttatacggatgatgaagatgatatctacaaggctaataacattgcctatgaagatgtggtcgggggagaagactggaacccagtagaggagaaaatagagagtcaaacccaggaagaggtgagagacagcaaagagaatatagaaaaaaatgaacaaatcaacgatgagatgaaacgctcagggcagcttggcatccaggaagaagatcttcggaaagagagtaaagaccaactctcagatgatgtctccaaagtaattgcctatttgaaaaggttagtaaatgctgcaggaagtgggaggttacagaatgggcaaaatggggaaagggccaccaggctttttgagaaacctcttgattctcagtctatttatcagctgattgaaatctcaaggaatttacagatacccccagaagacttaattgagatgctcaaaactggggagaagccgaatggatcagtggaaccggagcgggagcttgaccttcctgttgacctagatgacatctcagaggctgacttagaccatccagacctgttccaaaataggatgctctccaagagtggctaccctaaaacacctggtcgtgctgggactgaggccctaccagacgggctcagtgttgaggatattttaaatcttttagggatggagagtgcagcaaatcagaaaacgtcgtattttcccaatccatataaccaggagaaagttctgccaaggctcccttatggtgctggaagatctagatcgaaccagcttcccaaagctgcctggattccacatgttgaaaacagacagatggcatatgaaaacctgaacgacaaggatcaagaattaggtgagtacttggccaggatgctagttaaataccctgagatcattaattcaaaccaagtgaagcgagttcctggtcaaggctcatctgaagatgacctgcaggaagaggaacaaattgagcaggccatcaaagagcatttgaatcaaggcagctctcaggagactgacaagctggccccggtgagcaaaaggttccctgtggggcccccgaagaatgatgataccccaaataggcagtactgggatgaagatctgttaatgaaagtgctggaatacctcaaccaagaaaaggcagaaaagggaagggagcatattgctaagagagcaatggaaaatatgtaagctgctttcattaattaccctactttcattcctcccaccccaagcaaatcccaacatttctcttcagtgtgttgacttctatcctgttaacactgtaatatctttaaatgatgtacaggcagatgaaaccaggtcactggggagtctgcttcatttcctctgagctgttatcttgtgtatggatatgtgtaaatgttatgactccttgataaaaaatttattatgtccattattcaagaaagatatctatgactgtgtttaatagtatatctaatggctgtggcattgttgatgctcacatatgataaaaaagtgtcctataattctattgaaagtttttaatatttattgaattattttgttactgtctgtagtgttttgtggagtactggaccaaaaaaataaagcattataaatatatagttttatttataaggccttttctattgtgtgttttactgttgattaataaatgttatttctggacaaaaaaaaaaaaaaaa

SEQ ID NO: 4 - protein, 40 aa, Homo sapiens
APPGRPEAQPPPLSSEHKEPVAGDAVPGPKDGSAPEVRGA

SEQ ID NO: 5 - protein, 616 aa, Homo sapiens
MKALRLSASALFCLLLINGLGAAPPGRPEAQPPPLSSEHKEPVAGDAVPGPKDGSAPEVRGARNSEPQDEGELFQGVDPRALAAVLLQALDRPASPPAPSGSQQGPEEEAAEALLTETVRSQTHSLPAAGEPEPAAPPRPQTPENGPEASDPSEELEALASLLQELRDFSPSSAKRQQETAAAETETRTHTLTRVNLESPGPERVWRASWGEFQARVPERAPLPPPAPSQFQARMPDSGPLPETHKFGEGVSSPKTHLGEALAPLSKAYQGVAAPFPKARRAESALLGGSEAGERLLQQGLAQVEAGRRQAEATRQAAAQEERLADLASDLLLQYLLQGGARQRGLGGRGLQEAAEERESAREEEEAEQERRGGEERVGEEDEEAAEAAEAEADEAERARQNALLFAEEEDGEAGAEDKRSQEETPGHRRKEAEGTEEGGEEEDDEEMDPQTIDSLIELSTKLHLPADDVVSIIEEVEEKRNRKKKAPPEPVPPPRAAPAPTHVRSPQPPPPPPSARDELPDWNEVLPPWDREEDEVYPPGPYHPFPNYIRPRTLQPPSALRRRHYHHALPPSRHYPGREAQARHAQQEEAEAEERRLQEQEELENYIEHVLLRRP

SEQ ID NO: 6 - protein, 37 aa, Homo sapiens
GRPEAQPPPLSSEHKEPVAGDAVPGPKDGSAPEVRGA